(19) United States
(12) Patent Application Publication
(10) Pub. No.: US 2007/0238928 A1
(43) Pub. Date: Oct. 11, 2007

(54) Biopsy port for easy device passage

(75) Inventors: Luis J. Maseda, Natick, MA (US); William Lucas Churchill, Bolton, MA (US); Kurt Geitz, Sudbury, MA (US); Oscar Carrillo JR., Attleboro, MA (US); Matthew Whitney, Upton, MA (US); Adam Cohen, Arlington, MA (US); Roy H. Sullivan, Millville, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Boston Scientific Scimed, Inc., Maple Grove, MN (US)

(21) Appl. No.: 11/400,806

(22) Filed: Apr. 7, 2006

(51) Int. Cl. A61B 1/00

(52) U.S. Cl. 600/153

(57) ABSTRACT

A medical device includes a handle and a shaft having a working channel. A biopsy port at the exterior of the handle provides a path for instruments leading to the working channel. A conduit that runs through the handle for supplying fluids is also provided a path to the working channel. As compared between the path from the biopsy port to the working channel and the path from the conduit to the working channel, the path from the biopsy port to the working channel is the more direct path of the two.

### FIELD OF THE INVENTION

The present invention relates to medical devices in general, and to endoscopes in particular.

### BACKGROUND

As an alternative to performing more invasive medical procedures, many physicians are utilizing endoscopes and catheters to perform diagnostic and therapeutic procedures on internal tissues of patients. With this less invasive approach, a medical device, such as an endoscope or catheter, is advanced to a site of interest and the indicated procedure is performed. Most endoscopes and catheters have a flexible shaft that is advanced through the patient's anatomy until the distal tip reaches the tissue of interest. Once the tissue is reached, various other instruments may need to reach the tissue. To avoid multiple insertions of instruments, the handles of many endoscopes usually have a biopsy port through which a variety of instruments can be admitted. The biopsy port leads to a lumen in the shaft, commonly referred to as a working channel, which allows instruments to be guided through the shaft and to the tissue of interest. To conserve space within the shaft and to avoid multiple lumens, the working channel may also be used for the passage of various liquids and gases to the tissue of interest. For example, washing liquids, such as water or saline, air, or vacuum can be directed to the tissue of interest via the working channel. In order to allow for both instruments and fluids to pass from the endoscope handle to the working channel, the endoscope handle will typically include a “Y-connector” or other multi-way connector. Instruments inserted into the handle are directed by one proximal leg of the Y-connector into the working channel. One of the problems with a conventional Y-connector is that it can be difficult to guide the instruments into the working channel due to bends in the Y-connector. Therefore, there is a need for an improved mechanism for inserting tools into a working channel of an endoscope or other elongated medical device.

### SUMMARY

The present invention is related to a medical device in general, and an endoscope in particular, having a shaft with a lumen, or working channel, wherein the working channel is accessed from both a biopsy port and from a conduit through which fluids, gases, and/or vacuum may be applied. Both the biopsy port and the conduit lead into a common working channel. The biopsy port has the more direct path to the working channel, while the conduit has the less direct path to the working channel.

In one embodiment of the present invention, a common working channel is accessed through a Y-connector that includes a first proximal leg, a second proximal leg, and a third distal leg. Both proximal legs lead to the distal leg. One proximal leg is set substantially in line and coaxial with the distal leg, such that the centerlines of the proximal leg and the distal leg are generally aligned. The second proximal leg is set at an angle with respect to the first proximal leg and to the distal leg, such that the second proximal leg makes an acute angle with respect to the first proximal leg and an obtuse angle with respect to the distal leg. The first proximal leg is coupled to the biopsy port, which leads to the common working channel. The second proximal leg is coupled to a source of fluid/gas/vacuum, which leads to the working channel. In this configuration, a more direct path to the common working channel is from the biopsy port through the first proximal leg of the Y-connector. The biopsy port and the Y-connector may be fabricated from a flexible material, so that the port and Y-connector can conform to instruments that are inserted to reach the working channel.

In other embodiments, an endoscope handle can include a variety of accessories and attachments connected to the handle at or near the biopsy port for performing a variety of functions. Such accessories can include various devices to seal the biopsy port and to attach instruments to the handle so that they remain within the biopsy port.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of a representative handle of an endoscope including a biopsy port, in accordance with one embodiment of the present invention;

FIG. 2 is a diagrammatical illustration of the interior of a representative handle of an endoscope including a biopsy port and a Y-connector, in accordance with one embodiment of the present invention;

FIG. 3 is a diagrammatical illustration of a Y-connector for a medical device in accordance with another embodiment of the present invention;

FIG. 4 is a diagrammatical illustration of a representative handle of an endoscope including a biopsy port, in accordance with one embodiment of the present invention;

FIG. 5 is a diagrammatical illustration of a representative handle of an endoscope including a biopsy port, in accordance with one embodiment of the present invention;

FIG. 6 is a diagrammatical illustration of a biopsy port and a Y-connector, in accordance with one embodiment of the present invention;

FIG. 7 is a diagrammatical illustration of a biopsy port and a Y-connector, in accordance with one embodiment of the present invention;

FIG. 8 is a diagrammatical illustration of a representative handle of an endoscope including a biopsy port, in accordance with one embodiment of the present invention;

FIG. 9 is a diagrammatical illustration of a biopsy port and a Y-connector, in accordance with one embodiment of the present invention FIG. 10 is a diagrammatical illustration of a representative handle of an endoscope including a biopsy port, in accordance with one embodiment of the present invention;

FIG. 11 is a diagrammatical illustration of a representative handle of an endoscope including a biopsy port, in accordance with one embodiment of the present invention; and

FIG. 12 is a diagrammatical illustration of a biopsy port and a Y-connector, in accordance with one embodiment of the present invention.

### DETAILED DESCRIPTION

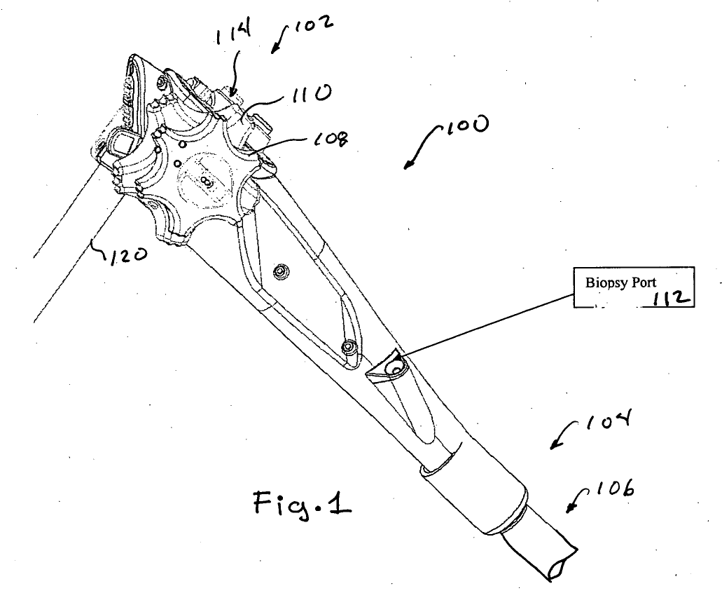

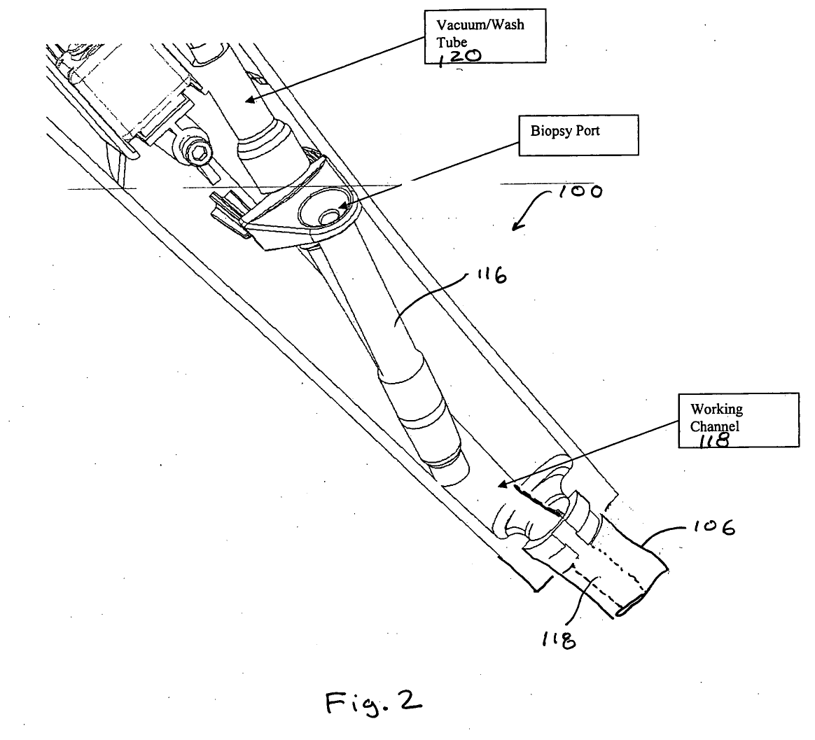

FIG. 1 is an illustration of an endoscope handle 100 including an outside biopsy port 112. The endoscope handle 100 has a proximal end 102 and a distal end 104. The distal end 104 is connected to an elongated shaft 106 of which only the proximal end is shown. The proximal end 102 of the handle 100 has steering knobs 108 and 110 that control steering wires or cables (not shown) that allow the shaft 106 to be steered in four directions. The shaft 106 includes a lumen, or working channel 118 (best seen in FIG. 2), which is interior to the shaft 106 and extends the length of the shaft 106 from the proximal end of the shaft 106 to the distal tip (not shown). The working channel 118 allows instruments and/or fluids to reach the distal tip of the shaft 106 for a variety of purposes. The biopsy port 112 permits instruments that are external to the handle 100 to be guided into the working channel 118 from an external location to eventually reach the distal tip of the shaft 106 where the instrument can be used to perform a medical procedure. The biopsy port 112 may be coated internally with a lubricious material, such as silicone or a fluorinated polymer, such as polytetrafluoroethylene, to permit instruments to slide more readily. Furthermore, in addition to the biopsy port 112, the Y-connector 116 and the working channel 118 can be coated with lubricious materials to facilitate instrument passage. The delivery of fluids, including saline or washing liquids, as well as the delivery of one or more of air, or vacuum can be applied to the working channel 118 such that they can reach the distal tip of the shaft 106. In one embodiment, the delivery of the various fluids, gases, or vacuum is controlled with the handle 100 via a plurality of buttons 114. Fluids, air, or vacuum are supplied from a fluid supply source (not shown) via a conduit or tubing 120, which is connected to the proximal end 102 of the handle 100.

FIG. 2 is an illustration of the interior of the endoscope handle 100 showing how both instruments and fluids/air/vacuum can pass through the handle 100 into the working channel 118 in accordance with one embodiment of the present invention. Not all of the components of the endoscope handle 100 are being shown for clarity and brevity. The handle 100 includes a Y-connector 116. The shaft 106, of which only the proximal connection to the handle 100 is illustrated, includes the working channel 118, disposed in the interior of the shaft 106. The working channel 118 has an opening at its proximal end to receive the various fluids, air, gas, or vacuum and instruments. The working channel 118 is accessible to external instruments via the biopsy port 112 at the exterior of the handle 100 and to fluids/air/gas/vacuum via the conduit 120 that passes through the handle 100. Both instruments and fluids originate from different locations, but are directed to the common proximal opening of the working channel 118. The Y-connector 116 is disposed at the distal end of the handle 100 and is in communication with the proximal opening of the working channel 118 for this purpose.

As seen in FIG. 3, the Y-connector 116 includes a first proximal leg 122, a second proximal leg 124, and a common distal leg 126. The first proximal leg 122, second proximal leg 124, and common distal leg 126 each has a lumen therein. The lumen of the common distal leg 126 is coupled to the working channel 118, either through a direct connection or an indirect connection via an additional lumen. Both lumens of the proximal legs 122, 124 lead to the lumen of the common distal leg 126. The first proximal leg 122 is generally aligned with respect to the common distal leg 126, such that the centerlines of the first proximal leg 122 and the common distal leg 126 form an angle of approximately 180°. The second proximal leg 124 is set at an angle with respect to the first proximal leg 122 and with respect to the common distal leg 126, such that the second proximal leg 124 makes an acute angle with respect to the first proximal leg 122 and an obtuse angle with respect to the common distal leg 126. The first proximal leg 122 leads from the biopsy port 112 to the working channel 118. The second proximal leg 124 leads from the conduit 120 to the working channel 118.

In the illustrated configuration, the more direct path through the Y-connector 116 to the working channel 118 is through the first proximal leg 122 of the Y-connector 116, which is the path from the biopsy port 112 to the working channel 118. The Y-connector 116 allows the introduction of an instrument into the working channel 118 from a position exterior to the handle 100. The instrument may be passed from the biopsy port 112 through the first proximal leg 122 of the Y-connector 116 and into the proximal opening of the working channel 118 within the shaft 106. The Y-connector 116 also allows the passage of fluids/air/vacuum from any one of a multitude of sources from the conduit 120 through the second proximal leg 124 of the Y-connector 116 to the proximal opening of the working channel 118. The second proximal leg 124 is connected to the fluids conduit or tubing 120, via a fluid tight connection such as a barbed end, for example, to which the conduit or tubing 120 is connected.

As indicated above, the delivery of liquids and/or gases is prompted by the actuation of one or more of the buttons 114 located on the handle 100. The second proximal leg 124 is oriented at an angle to the line defined by the centerlines of the first proximal leg 122 and the common distal leg 126. Therefore, fluids, gases, or vacuum that pass through the Y-connector 116 do not have a straight or direct path to the working channel 118. Since liquids and gases or vacuum are generally unaffected by the shape of any container or conduit through which they pass, the indirect path through the Y-connector 116 does not offer much resistance to their passing through the Y-connector 116. Instruments that are inserted into the working channel 118, although flexible, may nevertheless be easier for physicians to guide through the working channel 118 when the path from the biopsy port 112 to the working channel 118 is direct or as straight as possible.

As discussed above, the path from the biopsy port 112 to the working channel 118 is direct or, at least, of the two possible paths through the Y-connector 116, the path from the entrance of the biopsy port 112 to the working channel 118 is the more direct path through the Y-connector 116 to the working channel 118. An instrument, such as biopsy forceps, snare, optical fiber, etc., can be inserted through the biopsy port 112 and guided in a substantially direct path to the working channel 118 with less of a probability that the instrument will catch on a bend in the Y-connector 116.

In one embodiment, the biopsy port 112 may be fitted with a septum seal 128 covering the biopsy port's 112 opening. A septum seal 128 may be elastic to expand to cover and to seal around the outer periphery of any instrument that may be inserted through the septum seal 128 and into the biopsy port 112. Furthermore, when the biopsy port 112 is not in use, the opening through the septum seal 128 is closed and prevents the inadvertent or unintentional discharge of fluids to the outside. Even when in use, the septum seal 128 can provide a layer of protection against contact with bodily fluids that may be within the working channel 118 because the septum seal 128 will close around the outer periphery of any instrument inserted through the biopsy port 112. Further, the septum seal 128 provides an airtight seal in the Y-connector so that air does not enter through the biopsy port 112 and vacuum can reach the working channel.

In another embodiment, the Y-connector 116 or, at least, the proximal leg 122 or any other lumens from the proximal leg 122 that leads to the biopsy port 112, is formed from an elastomer that is flexible to bend with the bends in the instrument. Such elastomers may include materials known by the designations: Santoprene™, Hytrel™, Pellethane™, and Kraton™. Such elastomers may include one or a combination of the following polymers, block copolymers, and triblock copolymers, for example, a polypropylene, a polyester, a polyurethane, a polyisoprene, a styrene block copolymer, a poly(ethylene-propylene) block copolymer, a poly(styrene-butadiene-styrene) block copolymer, a poly(styrene-isoprene-styrene) block copolymer, a poly(styrene-ethylene-butylene-styrene) triblock copolymer, and a poly(styrene-ethylene-propylene-styrene) triblock copolymer.

Although the Y-connector 116 has been illustrated as a separate and distinct component, in another embodiment, an integral Y-connector can be formed as part of the conduit 120 through which fluids are transferred. In this embodiment, the conduit 120 through which fluids are transferred and the Y-connector 116 are one and the same component. Alternatively, the septum seal 128, biopsy port 112, and Y-connector 116 can all be integrally formed as a monolithic unit or any two of the three can be integrally formed as a monolithic unit. Further still, the Y-connector 116 may be integrally formed as part of the working channel 118. In this embodiment, the path from the biopsy port 112 to the working channel 118 is still the more direct path through the Y-connector 116.

Referring to FIG. 4, a portion of an endoscope handle 100 is illustrated, including a Y-connector 116, wherein the entrance to the biopsy port 112 is provided with a universal connector 150. The universal connector 150 may be used with a variety of attachments for a variety of purposes.

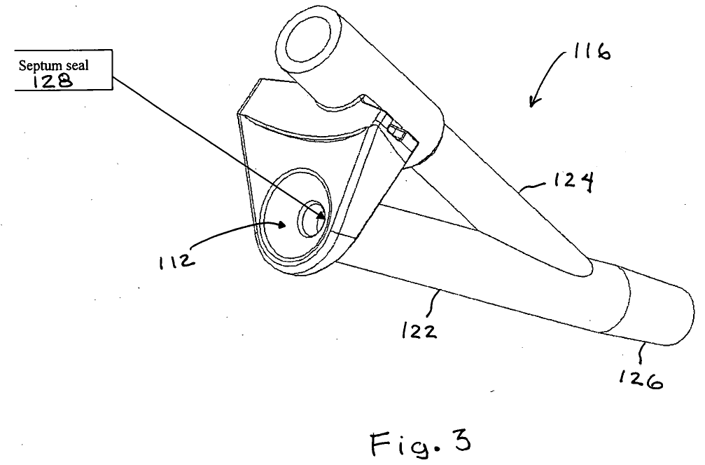

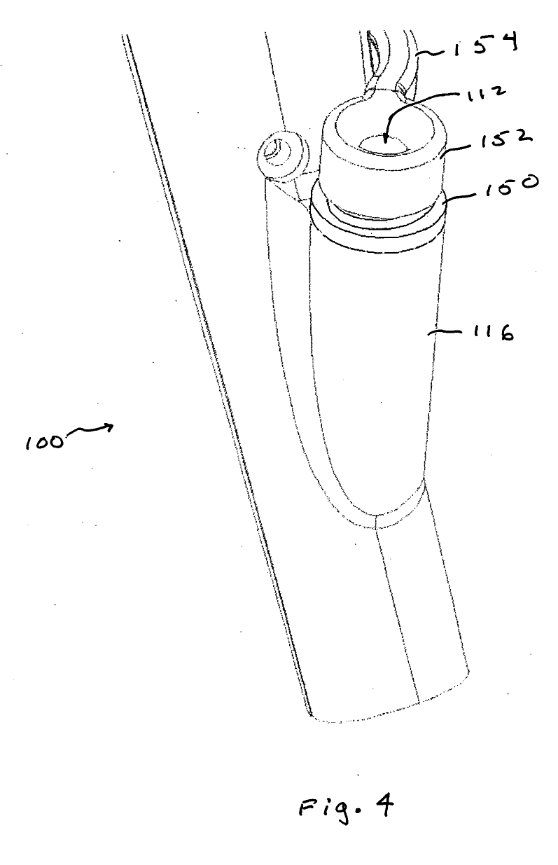

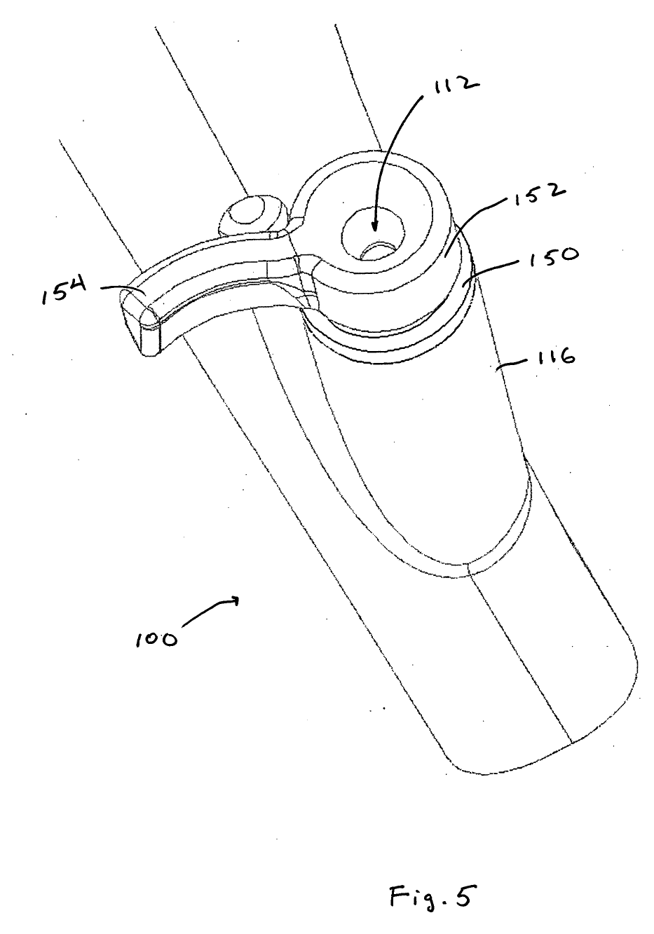

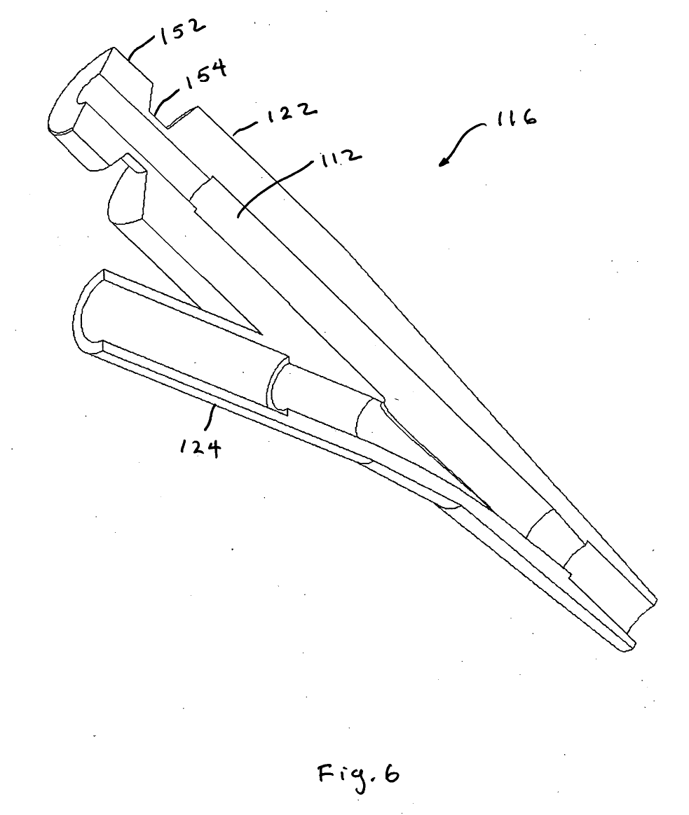

In one embodiment, the universal connector 150 is used for attaching a selectively sealable cap 152. The cap 152 includes a bore therethrough in the center that allows access to the biopsy port 112. The cap 152 includes a lever 154 to rotate the cap 152, thereby selectively closing and opening the access opening to the biopsy port 112. The lever 154 is one implementation; however, the cap 152 may have a non-skid gripping surface around the perimeter that provides an alternative means for gripping the cap 152 to rotate it. In one embodiment, the universal connector 150 includes exterior threads (not shown), while the cap 152 includes interior mating threads. In this embodiment, rotation of the cap 152 will result in either tightening or releasing the cap 152. In FIG. 5, the lever 154 has been rotated approximately 180°, thereby selectively closing access to the biopsy port 112. In the closed position, the biopsy port 112 can withstand aspiration and pressure.

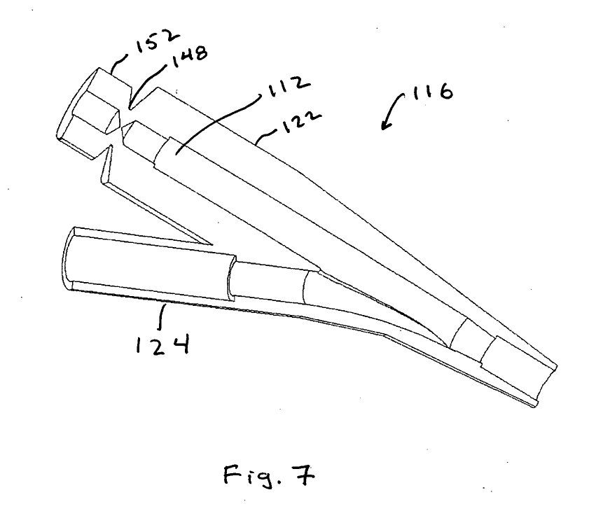

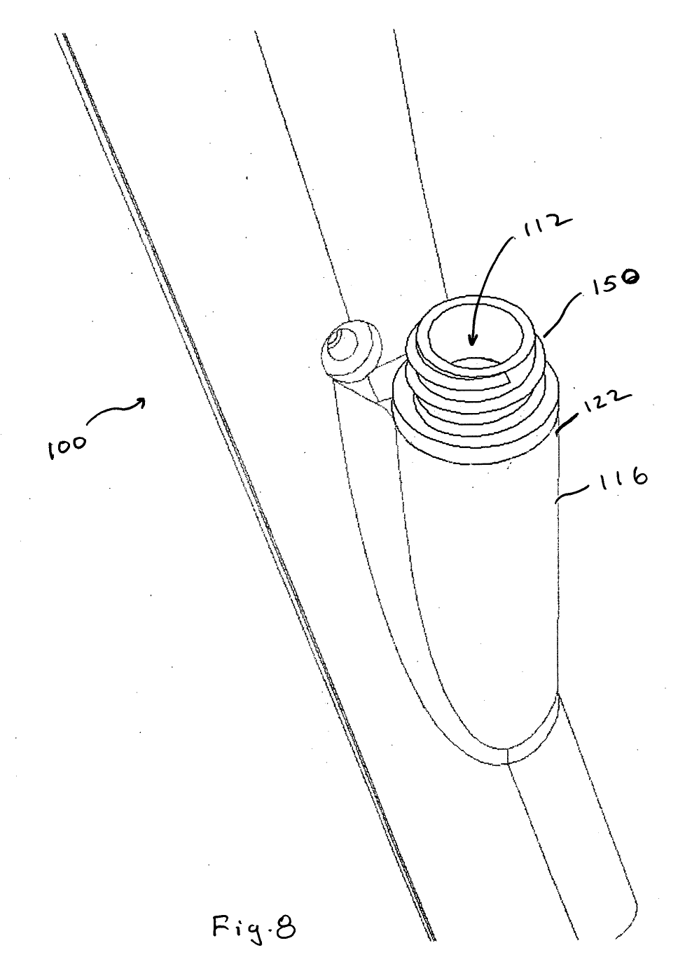

FIG. 6 is a cross-sectional illustration of the Y-connector 116 with the cap 152. The biopsy port 112 is provided in the proximal leg 122 of the Y-connector 116. The cap 152 is attached at the entrance of the biopsy port 112. A compliant material 148 is interposed between the cap 152 and the proximal leg 122. The universal connector 150 is not being shown for clarity. The compliant material 148 can be fabricated integral with the proximal leg 122 of the Y-connector 116. Alternatively, the compliant material 148 can be an added component. The compliant material 148 can be silicone or an elastomer of the type mentioned above. As described above, the cap 152 can be rotated and such rotation can lead to compression of the compliant material 148. In FIG. 7, the compliant material 148 has been compressed by rotating the cap 152, which causes the compliant material 148 to bulge around the bore of the biopsy port 112, thus sealing the entrance of the biopsy port 112. If an instrument is located in the biopsy port 112, the compliant material will seal around the instrument and lock the instrument in place. Thus, the cap 152 and the compliant material 148 provide an example of means for selectively sealing the biopsy port 112.

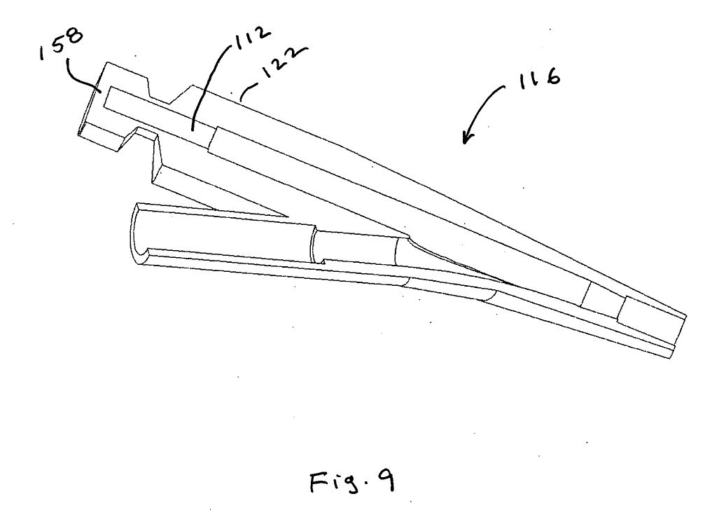

In FIG. 8, the universal connector 150 is illustrated. The universal connector 150 can be integral to the Y-connector 116. The universal connector 150 is attached to the proximal leg 122. In one embodiment, the universal connector 150 includes threads. However, other embodiments may utilize a snap-fit device, such as individually disposed fingers having barbs at the ends thereof that can be snap-fitted to a variety of attachments. Such attachments may include corresponding connectors on any medical device that can be used with the biopsy port 112. For example, a medical device can have a collar with internal threads matching the threads of the universal connector 150. Such collar can be threaded onto the universal connector 150 to rigidly fix an instrument in the biopsy port 112 of the endoscope handle 100, thus, leaving the physician with a free hand to perform another function.

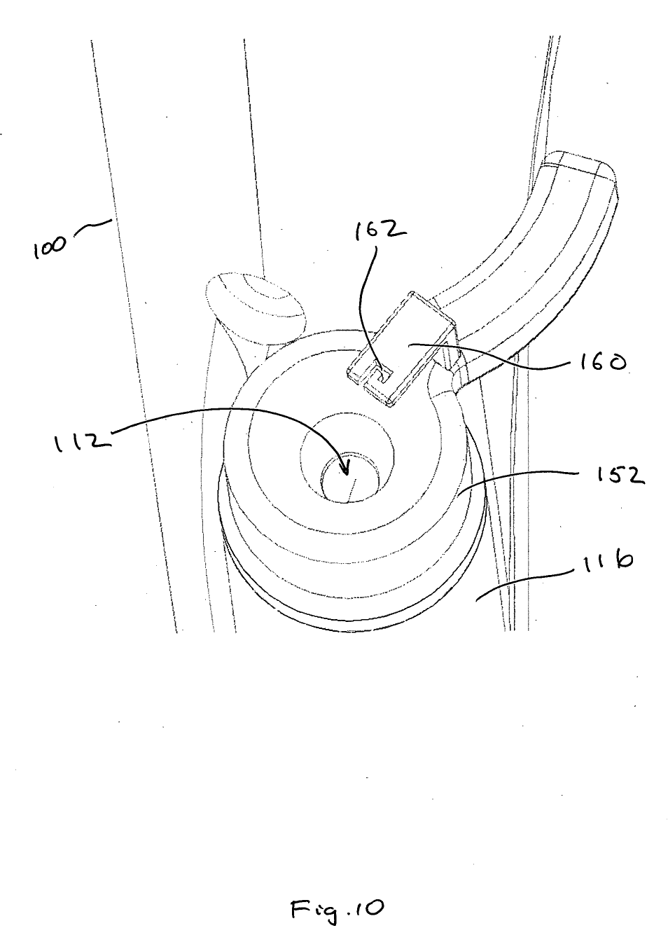

In FIG. 9, another embodiment of the Y-connector 116 includes a membrane 158 at the entrance of the biopsy port 112. The membrane 158 can be provided as an attachment to the universal port 150 (shown in FIG. 8) or the membrane 158 can be integral with the proximal leg 122 of the Y-connector 116. The membrane 158 is for maintaining a seal around the entrance of the biopsy port 112. The closed membrane 158 maintains pressure and aspiration and allows a tool to be locked in place. The membrane 158 can be punctured or pre-cut with a slit, such that the membrane 158 can remain closed even when an instrument is inserted therethrough. The slit configuration could be a line, cross, star, or reverse star to minimize the friction created during instrument passage while still allowing the seal to be closed and maintain insufflation pressure when no tool is present.

An advantage of the universal connector 150 is the elimination of straps that are being used to hold devices in the biopsy port 112. With a universal connector 150, in accordance with the present invention, the use of straps and external hold-downs is eliminated. The universal connector 150 can be integral with the endoscope handle 100. The universal connector 150 can be used with a variety of cooperating attachments. Such attachments can include, but are not limited to, a selectively opening and closing cap, a sealed membrane, and any medical instrument that can be inserted through the biopsy port 112. A seal at the biopsy port 112 allows for both insufflation and aspiration with the endoscope. Furthermore, the cap 152 is selectively tightened to provide greater or lesser sealing ability. For example, the tightening of the cap 152 can adjusted to provide levels of compression of the compliant material 148 depending upon the medical instrument and the medical procedure requirements.

FIG. 10 is an illustration of another embodiment in accordance with the present invention. The endoscope handle 100 includes a rigid member 160, rigidly attached or affixed to the endoscope handle 100. In one embodiment, the rigid member 160 is connected to the cap 152 which is further connected to the Y-connector 116 at the biopsy port 112. The rigid member 160 has an arm extending above and/or adjacent to the biopsy port 112. The rigid member 160 includes a “J” shaped cutout 162 which is provided at the end of the rigid member 160. The “J” shaped cutout 162 acts as a locking feature for a medical device, such as a guidewire, to lock the guidewire at the biopsy port 112 when removing or exchanging catheters. In contrast to conventional endoscope handles, a rigid member 160 is provided on the endoscope handle 100 to eliminate the need for straps or tie-downs to secure instruments in the biopsy port 112.

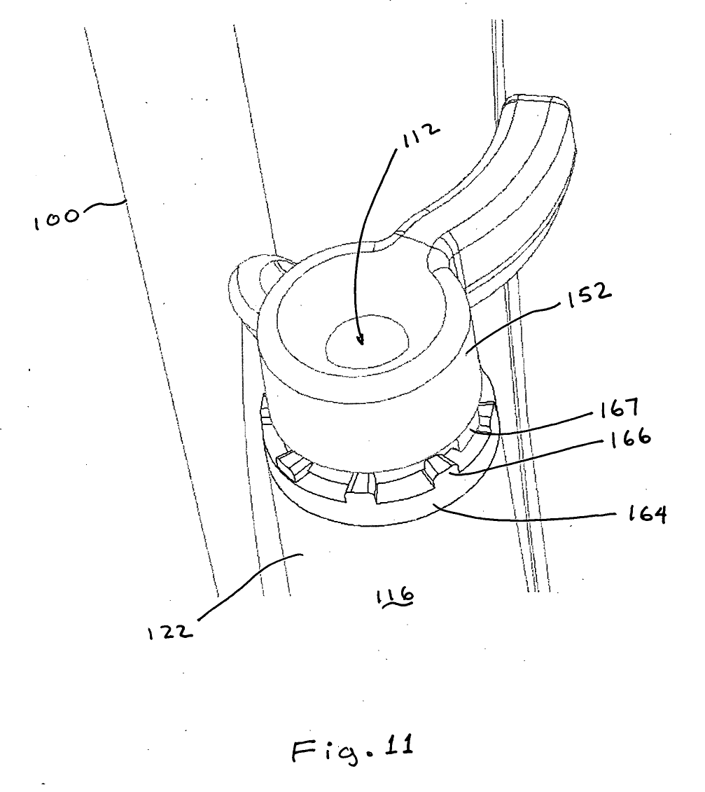

FIG. 11 is an illustration of another embodiment in accordance with the present invention. The endoscope handle 100 includes the universal connector 150 (not shown) located on leg 122 of the Y-connector 116. The endoscope handle 100 includes the cap 152, as described above, including the compliant material 148 (not shown) that seals the biopsy port 112. A circular detent 166 is provided between the cap 152 and the leg 122 of the Y-connector 116. The circular detect 164 includes upwardly projecting teeth 166 disposed around the circumference on the upper surface of the circular detent 164. The teeth 166 include generally rounded edges. The cap 152 can include downwardly projecting teeth 167 or notches (not shown) on the underside of the cap 152 which pass over the teeth 166 of the circular detent 164 when the cap 152 is rotated to compress the compliant material 148. The intermeshing of the teeth 167 of cap 152 and the teeth 166 of circular detent 164 provide the ability to hold and maintain the amount of rotation of cap 152 at any degree, and thus, to regulate the amount of compression of the compliant material 148 on the biopsy port 112 or on any instrument that is within the biopsy port 112. For example, the cap 152 can be rotated counterclockwise, thereby producing a “clicking” noise as the teeth 167 of cap 152 pass over the teeth 166 of the circular detent 164. When released, the cap 152 is prevented from moving by a certain amount of resistance that is required for the teeth 167 of cap 152 to pass over the teeth 166 of the circular detent 164. Thus, the cap 152, the compliant material 148, and the circular detent 164 provide a means for regulating the amount of sealing at the biopsy port 112 in discrete steps.

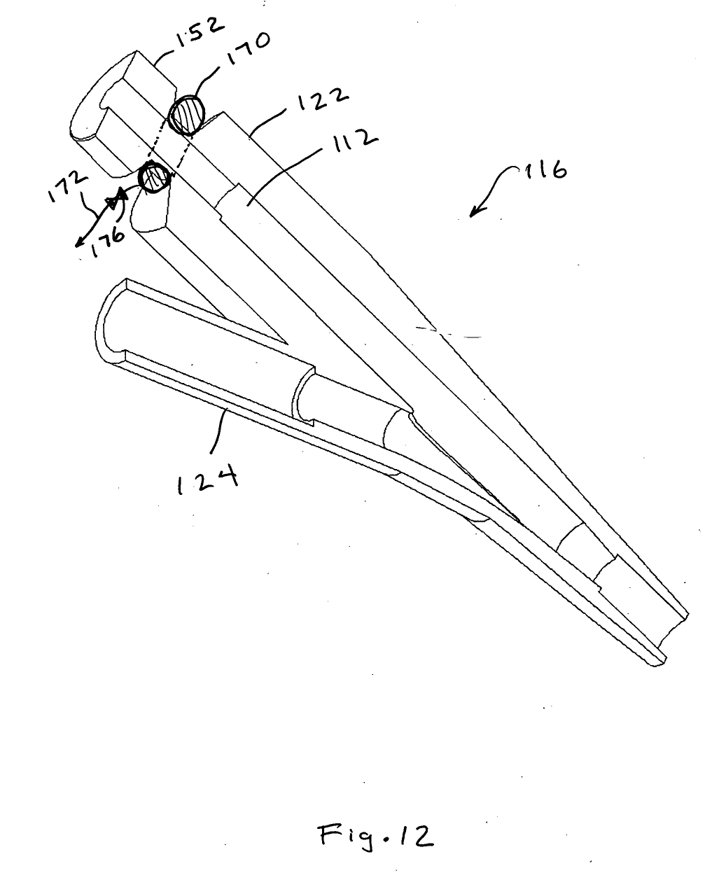

FIG. 12 is an illustration of another embodiment in accordance with the present invention. Leg 122 of Y-connector 116 includes the cap 152 having a “donut-shaped” inflatable bladder 170. The bladder 170 is placed around the biopsy port 112 so that the center hole of the bladder 170 surrounds the biopsy port 112. The bladder 170 is connected to inflation line 172. A valve 176 is provided in the line 172. The bladder 170 can be inflated by introducing any pressurized fluid from the line 172. For example, the bladder 170 can be inflated simultaneously with operation of an insufflation process through actuation via the endoscope handle 100 (not shown). The valve 176 opens under a set pressure to inflate the bladder 170 to create a seal around an instrument located in the biopsy port 112 in order to carry out the insufflation procedure on the patient. Alternatively, if there is no instrument in the biopsy port 112, the bladder 170 can inflate sufficiently to completely seal the biopsy port 112. As the insufflation pressure is decreased, the valve 176 allows pressure to be released from the bladder 170, thus opening the biopsy port 112 and/or releasing the instrument that is within the biopsy port 112. If instrument removal is necessary, the valve 176 would allow all pressure in the bladder 170 to bleed out, therefore, rendering the instrument free to be removed from the biopsy port 112. In addition to insufflation pressure using an air source, the bladder 170 can be inflated with a liquid from a bolus wash source. Alternatively, any other source of pressure, whether liquid or gas, can be used to inflate the bladder 170.

While illustrative embodiments have been illustrated and described, it will be appreciated that various changes can be made therein without departing from the spirit and scope of the invention.

The embodiments of the invention in which an exclusive property or privilege is claimed are defined as follows:

1. A medical device, comprising:
(a) a shaft having a working channel disposed within the shaft through which instruments and fluids and/or vacuum are passed to reach a distal end of the shaft;
(b) a handle connected to the proximal end of the shaft, wherein the handle includes a biopsy port through which instruments are admitted to reach the working channel; and
(c) a Y-connector within the handle that includes a first leg, a second leg, and a third leg, wherein the first leg of the Y-connector is coupled to the biopsy port, the second leg of the Y-connector is coupled to a source of fluids and/or vacuum, and the third leg of the Y-connector is coupled to the working channel, wherein the Y-connector includes a path through the first leg from the biopsy port to the working channel which is more direct than a path through the second leg to the working channel.

2. The medical device of claim 1, wherein the path from the biopsy port to the working channel is relatively straight through the first and third legs of the Y-connector.

3. The medical device of claim 1, wherein the Y-connector is made from a flexible material.

4. The medical device of claim 1, wherein the Y-connector is made from a thermoplastic elastomer.

5. The medical device of claim 1, wherein the Y-connector is made from a polypropylene, a polyester, a polyurethane, a polyisoprene, a styrene block copolymer, a poly(ethylene-propylene) block copolymer, a poly(styrene-butadiene-styrene) block copolymer, a poly(styrene-isoprene-styrene) block copolymer, a poly(styrene-ethylene-butylene-styrene) triblock copolymer, and a poly(styrene-ethylene-propylene-styrene) triblock copolymer.

6. The medical device of claim 1, further comprising a seal to cover the opening of the biopsy port to block fluids from exiting through the biopsy port.

7. The medical device of claim 1, wherein the path to the working channel through second and third legs of the Y-connector includes an angle bend.

8. The medical device of claim 1, wherein the first, second, and third leg each has a lumen therein and the lumens of the first leg and third leg are substantively in-line with each other and the second leg joins the Y-connector at an angle with respect to the aligned first and third legs.

9. A medical device, comprising:
(a) a handle having a distal end;
(b) a shaft having a proximal end and a working channel, wherein the proximal end of the shaft is connected to the distal end of the handle;
(c) a biopsy port provided at the exterior of the handle, wherein the biopsy port provides an entrance to a path leading to the working channel; and
(d) a conduit that provides a path between the biopsy port and the working channel and provides a fluid and/or vacuum connection to the working channel, wherein as compared between the path from the biopsy port to the working channel and the fluid and/or vacuum path to the working channel, the path from the biopsy port to the working channel is the more direct path of the two.

10. The medical device of claim 9, comprising a Y-connector having a first leg, a second leg and a third leg, each leg having a lumen therein, whereby the first leg is coupled to the biopsy port and the third leg is coupled to the working channel such that the lumens of the first leg and third leg are substantively in-line with each other and whereby the second leg joins the Y-connector at an angle with respect to the aligned first and third legs.

11. A medical device, comprising:
(a) a handle;
(b) a shaft having a working channel therein, wherein the shaft is connected to the handle;
(c) a biopsy port provided at the exterior of the handle; and
(d) a flexible lumen that joins the biopsy port to the working channel.

12. The medical device of claim 11, wherein the flexible lumen that joins the biopsy port to the working channel includes a Y-connector.

13. The medical device of claim 12, wherein the Y-connector is made from a thermoplastic elastomer.

14. The medical device of claim 11, comprising a Y-connector having a first leg, a second leg, and a third leg, each leg having a lumen therein, whereby the first leg is coupled to the biopsy port and the third leg is coupled to the working channel such that the lumens of the first leg and third leg are substantively in-line with each other and whereby the second leg joins the Y-connector at an angle with respect to the aligned first and third legs.

15. The medical device of claim 11, wherein the flexible lumen is made from a polypropylene, a polyester, a polyurethane, a polyisoprene, a styrene block copolymer, a poly(ethylene-propylene) block copolymer, a poly(styrene-butadiene-styrene) block copolymer, a poly(styrene-isoprene-styrene) block copolymer, a poly(styrene-ethylene-butylene-styrene) triblock copolymer, or a poly(styrene-ethylene-propylene-styrene) triblock copolymer.

16. A medical device, comprising:
(a) a handle having a distal end;
(b) a shaft having a proximal end and a working channel, wherein the proximal end of the shaft is connected to the distal end of the handle;
(c) a biopsy port provided on the handle, wherein the biopsy port provides an entrance to a path leading to the working channel;
(d) a conduit providing a path for fluids and/or vacuum leading to the working channel; and
(e) a common lumen that connects both the biopsy port and the conduit to the working channel, wherein the path from the conduit to the common lumen defines an acute angle with respect to the path from the biopsy port to the common lumen, and wherein the path from the conduit to the common lumen defines an obtuse angle with respect to the path from the common lumen to the working channel.

17. A medical device, comprising:
a handle connected to a distal shaft, wherein the shaft includes a lumen therein; and
a port on the handle for access to the lumen, wherein the port includes a universal connector used for attaching an accessory at or to the port.

18. The medical device of claim 17, wherein the accessory comprises a cap for selectively sealing the port.

19. The medical device of claim 17, wherein the accessory comprises a cap for selectively sealing the port that includes compliant material that is compressed by the cap to seal the port.

20. The medical device of claim 17, wherein the accessory is a locking device attached to an instrument that is inserted into the biopsy port so that the locking device attaches to the universal connector to lock the instrument in the biopsy port.

21. The medical device of claim 17, wherein the accessory is a membrane that seals the port.

22. A medical device, comprising:
(a) a handle connected to a distal shaft, wherein the distal shaft includes a lumen therein; and
(b) a port on the handle for access to the lumen, wherein the handle includes a rigidly attached member for securing an instrument within the port.

23. The medical device of claim 22, wherein the instrument is a guidewire locking device.

24. The medical device of claim 22, wherein the rigidly attached member includes a cutout for securing an instrument within the port.

25. A medical device, comprising:
(a) a handle connected to a distal shaft, wherein the shaft includes a lumen therein; and
(b) a port on the handle for access to the lumen, wherein the port includes means for selectively sealing the port.

26. The medical device of claim 25, wherein the means for selectively sealing the port comprises a cap and a compliant material, wherein rotation of the cap compresses the compliant material to seal the port.

27. The medical device of claim 25, wherein the sealing of the port can be regulated in discrete steps.

28. The medical device of claim 27, wherein the means for selectively sealing the port comprises a cap, a compliant material, and a circular detent disposed in communication with the cap, wherein rotation of the cap compresses the compliant material, and the circular detent resists rotation of the cap in discrete steps.

29. The medical device of claim 28, wherein the cap comprises teeth which pass over teeth on the circular detent.

30. A medical device, comprising:
(a) a handle connected to a distal shaft, wherein the shaft includes a lumen therein;
(b) a port on the handle for access to the lumen; and
(c) an inflatable bladder at or in the port.

31. The medical device of claim 30, wherein the inflatable bladder is connected to a source of pressure for inflating the bladder to seal the port.

32. The medical device of claim 30, wherein the inflatable bladder can seal around an instrument inserted within the port.

33. The medical device of claim 30, comprising a valve which permits the inflation of the bladder and deflation of the bladder according to the pressure in a line that supplies pressure to the bladder.
